(19) United States
(12) Patent Application Publication
(10) Pub. No.: US 2025/0160723 A1
(43) Pub. Date: May 22, 2025

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventors: Kosuke NISHIHARA, Tokyo (JP); Yuan Luo, Tokyo (JP); Osamu Hisamatsu, Tokyo (JP); Mitsuru Noma, Tokyo (JP)

(73) Assignee: NEC Corporation, Minato-ku, Tokyo (JP)

(21) Appl. No.: 18/839,770

(22) PCT Filed: Mar. 28, 2022
(86) PCT No.: PCT/JP2022/014796

§ 371 Date: Aug. 20, 2024

(51) Int. Cl. A61B 5/346; G06N 20/00

(52) U.S. Cl. CPC A61B 5/346; G06N 20/00

(57) ABSTRACT

In order to generate training data for evaluating a disease from an electrocardiogram with higher accuracy, an information processing apparatus (1) includes: an acquisition section (11) for acquiring a plurality of unit waveforms which have been obtained by dividing a waveform indicated by electrocardiogram data; an extraction section (12) for extracting, using a model which has been trained with use of a certain group derived from the plurality of unit waveforms acquired by the acquisition section (11), one or more unit waveforms from another group derived from the plurality of unit waveforms; and a training data generation section (13) for generating training data including the one or more unit waveforms which have been extracted by the extraction section (12).

### TECHNICAL FIELD

The present invention relates to an information processing apparatus, an information processing method, and an information processing program.

### BACKGROUND ART

Attempts have been made to automatically predict a disease from waveform data of an electrocardiogram. For example, Non-patent Literature 1 discloses a method in which a single-cycle waveform is cut out from an electrocardiogram waveform, and binary classification is carried out to determine whether the electrocardiogram waveform is within a normal range or there is any abnormal finding, with use of a convolutional neural network model which has undergone machine learning using, as training data, a two-dimensional waveform image obtained by superimposing waveforms for respective cut-out cycles.

### CITATION LIST

### Non-Patent Literature

### [Non-Patent Literature 1]

Senami Furubayashi, et al., “A study on normal abnormal determination of electrocardiogram waveform using deep learning”, The Japanese Society for Artificial Intelligence, Materials of Special interest group (Type 2) 2018 (AIMED-005), 05, 2018

### SUMMARY OF INVENTION

### Technical Problem

Waveform data of an electrocardiogram includes waveforms in a plurality of cycles. However, not all cycles of waveforms indicate characteristics of a disease. Therefore, in the technique disclosed in Non-patent Literature 1, there is a possibility that a waveform in which a characteristic of a disease does not appear among waveforms in a plurality of cycles included in an electrocardiogram waveform may be learned as a characteristic of a disease. As a result, there is a problem that the technique described in Non-patent Literature 1 cannot accurately evaluate a disease.

An example aspect of the present invention is accomplished in view of the above problem, and an example object thereof is to provide a technique for generating training data for evaluating a disease from an electrocardiogram with higher accuracy.

### Solution to Problem

An information processing apparatus in accordance with an example aspect of the present invention includes: an acquisition means for acquiring a plurality of unit waveforms which have been obtained by dividing a waveform indicated by electrocardiogram data; an extraction means for extracting, using a model which has been trained with use of a certain group derived from the plurality of unit waveforms acquired by the acquisition means, one or more unit waveforms from another group derived from the plurality of unit waveforms; and a training data generation means for generating training data including the one or more unit waveforms which have been extracted by the extraction means.

An information processing method in accordance with an example aspect of the present invention includes: acquiring, by at least one processor, a plurality of unit waveforms which have been obtained by dividing a waveform indicated by electrocardiogram data; extracting, by the at least one processor, using a model which has been trained with use of a certain group derived from the plurality of unit waveforms, one or more unit waveforms from another group derived from the plurality of unit waveforms; and generating, by the at least one processor, training data including the one or more unit waveforms which have been extracted.

An information processing program in accordance with an example aspect of the present invention causes a computer to carry out: an acquisition process of acquiring a plurality of unit waveforms which have been obtained by dividing a waveform indicated by electrocardiogram data; an extraction process of extracting, using a model which has been trained with use of a certain group derived from the plurality of unit waveforms acquired in the acquisition process, one or more unit waveforms from another group derived from the plurality of unit waveforms; and a training data generation process of generating training data including the one or more unit waveforms which have been extracted in the extraction process.

### Advantageous Effects of Invention

According to an example aspect of the present invention, it is possible to generate training data for evaluating a disease from an electrocardiogram with higher accuracy.

### BRIEF DESCRIPTION OF DRAWINGS

FIG. 1 is a block diagram illustrating a configuration of an information processing apparatus in accordance with a first example embodiment.

FIG. 2 is a flowchart illustrating a flow of an information processing method in accordance with the first example embodiment.

FIG. 3 is a block diagram illustrating a configuration of an information processing apparatus in accordance with a second example embodiment.

FIG. 4 is a flowchart illustrating a flow of an information processing method in accordance with the second example embodiment.

FIG. 5 is a flowchart illustrating a flow of process example 1 of an extraction process.

FIG. 6 is a flowchart illustrating a flow of process example 2 of the extraction process.

FIG. 7 is a diagram exemplifying unit waveforms extracted in process example 2.

FIG. 8 is a flowchart illustrating a flow of process example 4 of the extraction process.

FIG. 9 is a diagram exemplifying unit waveforms extracted in process example 4.

FIG. 10 is a diagram illustrating an example of a training data generation process carried out by a training data generation section.

FIG. 11 is a diagram illustrating a display screen example which is displayed on a first display section.

FIG. 12 is a diagram illustrating a screen display example which is displayed on a second display section.

FIG. 13 is a diagram illustrating an example of a computer which executes instructions of a program that is software realizing functions of apparatuses in accordance with example embodiments of the present invention.

### EXAMPLE EMBODIMENTS

### First Example Embodiment

The following description will discuss a first example embodiment of the present invention in detail, with reference to the drawings. The present example embodiment is a basic form of example embodiments described later.

### <Configuration of Information Processing Apparatus 1>

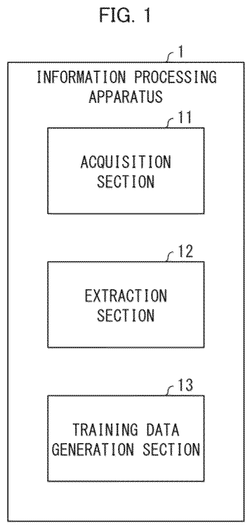

The following description will discuss a configuration of an information processing apparatus 1 in accordance with the present example embodiment, with reference to FIG. 1. FIG. 1 is a block diagram illustrating a configuration of the information processing apparatus 1. The information processing apparatus 1 is an apparatus for extracting training data for machine learning from electrocardiogram data. Here, the electrocardiogram data is data which records a change in electrical activity of myocardium over time. The electrocardiogram data is, for example, a 12-lead electrocardiogram or an esophageal lead electrocardiogram. Note that the electrocardiogram data is not limited to the example described above, and may be data representing another electrocardiogram.

The information processing apparatus 1 includes an acquisition section 11, an extraction section 12, and a training data generation section 13.

### (Acquisition Section 11)

The acquisition section 11 acquires a plurality of unit waveforms which are obtained by dividing a waveform indicated by electrocardiogram data. The unit waveform is a waveform which is a unit of evaluation. The unit waveform may be composed of one wavelength or two wavelengths. The unit waveform is, for example, a waveform in each section obtained by dividing a waveform indicated by electrocardiogram data by peak values (R waves). A method of dividing a waveform indicated by electrocardiogram data into unit waveforms is not limited to the method of dividing by peak values, and may be another method. The unit waveform may be, for example, a waveform obtained by detecting R waves from a waveform indicated by electrocardiogram data and cutting out the waveform based on cycles identified from detection intervals of the R waves (e.g., a section in which an R wave appears at a position that is ⅖ from the start and ⅗ from the end).

(Extraction section 12)

The extraction section 12 extracts, using a model which has been trained with use of a certain group derived from the plurality of unit waveforms acquired by the acquisition section 11, one or more unit waveforms from another group derived from the plurality of unit waveforms. Here, the model is a model which is used to extract a unit waveform as training data from a plurality of unit waveforms and which is generated by machine learning. A method of machine learning of the model is not limited. For example, a decision tree-based method, a linear regression method, or a neural network method may be used, or two or more of these methods may be used. An input into the model is, for example, data representing one or more unit waveforms. An output of the model is, for example, a label indicating whether or not being a disease or an evaluation value related to a disease.

More specifically, for example, the extraction section 12 extracts true-positive and true-negative unit waveforms from the foregoing another group. For example, the extraction section 12 extracts, from the foregoing another group, a unit waveform for which an output obtained by inputting the foregoing another group into the model satisfies a predetermined condition. For example, the predetermined condition is as follows:
an evaluation value output by a model is equal to or greater than a predetermined threshold; or
an evaluation value is highest among unit waveforms included in a single piece of electrocardiogram data.
   For example, the extraction section 12 may identify unit waveforms which are candidates for extraction based on outputs obtained by inputting the foregoing another group into the model, and extract a unit waveform which has been selected based on user operation from the identified unit waveforms. The user operation is operation with which a user selects a unit waveform. Examples of the user operation include operation on input apparatuses such as a mouse, a keyboard, a touch panel, an audio input apparatus, and an eye-controlled input apparatus.

Note that the method in which the extraction section 12 extracts one or more unit waveforms from a plurality of unit waveforms is not limited to the example described above, and the extraction section 12 may extract one or more unit waveforms by another method. For example, the extraction section 12 may extract a unit waveform based on uncertainty of an evaluation result which is obtained using Bayesian inference for deep learning, rather than an evaluation value.

### (Training Data Generation Section 13)

The training data generation section 13 generates training data including one or more unit waveforms which have been extracted by the extraction section 12. The training data includes, for example, a plurality of sets each including one or more unit waveforms and labels each indicating whether or not being a disease.

The training data generated by the training data generation section 13 is used, for example, for training an evaluation model. The evaluation model may include a model which has been used by the extraction section 12 to extract one or more unit waveforms, or may be a model different from that model. The evaluation model is, for example, a model for evaluating whether or not being a disease. An input into the evaluation model is, for example, data representing one or more unit waveforms. An output of the evaluation model includes, for example, a label indicating whether or not being a disease or an evaluation value related to a disease. A method of machine learning of the evaluation model is not limited. For example, a decision tree-based method, a linear regression method, or a neural network method may be used, or two or more of these methods may be used.

### <Example Advantage of Information Processing Apparatus 1>

As described above, the information processing apparatus 1 in accordance with the present example embodiment employs the configuration of including: the acquisition section 11 for acquiring a plurality of unit waveforms which have been obtained by dividing a waveform indicated by electrocardiogram data; the extraction section 12 for extracting, using a model which has been trained with use of a certain group derived from the plurality of unit waveforms acquired by the acquisition section 11, one or more unit waveforms from another group derived from the plurality of unit waveforms; and the training data generation section 13 for generating training data including the one or more unit waveforms which have been extracted by the extraction section 12. Therefore, according to the information processing apparatus 1 in accordance with the present example embodiment, it is possible to bring about an example advantage of generating training data for evaluating a disease from an electrocardiogram with higher accuracy.

### <Information Processing Program>

The functions of the foregoing information processing apparatus 1 can also be implemented by a program. The information processing program in accordance with the present example embodiment causes a computer to carry out: an acquisition process of acquiring a plurality of unit waveforms which have been obtained by dividing a waveform indicated by electrocardiogram data; an extraction process of extracting, using a model which has been trained with use of a certain group derived from the plurality of unit waveforms acquired in the acquisition process, one or more unit waveforms from another group derived from the plurality of unit waveforms; and a training data generation process of generating training data including the one or more unit waveforms which have been extracted in the extraction process.

### <Flow of Information Processing Method S10>

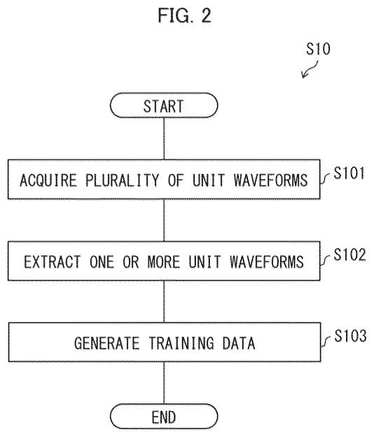

The following description will discuss a flow of an information processing method S10 in accordance with the present example embodiment, with reference to FIG. 2. FIG. 2 is a flowchart illustrating the flow of the information processing method S10. Steps of the information processing method S10 may be carried out by a processor included the information processing apparatus 1 or by a processor included in another apparatus. Alternatively, the steps may be carried out by processors provided in respective different apparatuses.

In step S101, at least one processor acquires a plurality of unit waveforms which are obtained by dividing a waveform indicated by electrocardiogram data. In step S102, the at least one processor extracts, using a model which has been trained with use of a certain group derived from the plurality of unit waveforms acquired in step S101, one or more unit waveforms from another group derived from the plurality of unit waveforms. In step S103, the at least one processor generates training data including the one or more unit waveforms which have been extracted in step S102.

### <Example Advantage of Information Processing Method S10>

As described above, the information processing method S10 in accordance with the present example embodiment employs the configuration of including: acquiring, by at least one processor, a plurality of unit waveforms which have been obtained by dividing a waveform indicated by electrocardiogram data; extracting, by the at least one processor, using a model which has been trained with use of a certain group derived from the plurality of acquired unit waveforms, one or more unit waveforms from another group derived from the plurality of unit waveforms; and generating, by the at least one processor, training data including the one or more unit waveforms which have been extracted. Therefore, according to the information processing method S10 in accordance with the present example embodiment, it is possible to bring about an example advantage of generating training data for a disease evaluating from an electrocardiogram with higher accuracy.

### Second Example Embodiment

The following description will discuss a second example embodiment of the present invention in detail, with reference to the drawings. The same reference numerals are given to constituent elements which have functions identical with those described in the first example embodiment, and descriptions as to such constituent elements are not repeated.

### <Configuration of Information Processing Apparatus 1A>

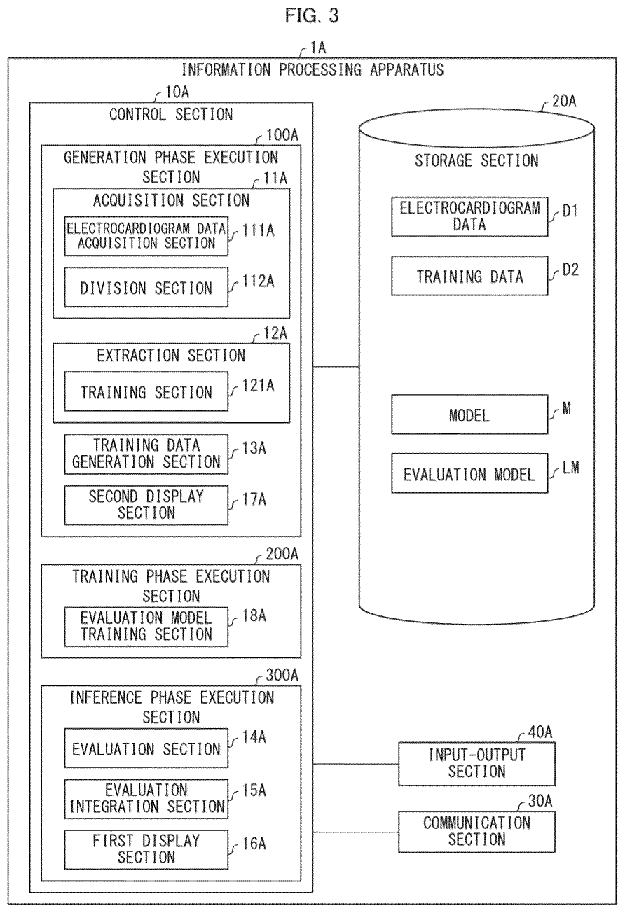

FIG. 3 is a block diagram illustrating a configuration of an information processing apparatus 1A in accordance with the present example embodiment. As illustrated in FIG. 3, the information processing apparatus 1A includes a control section 10A, a storage section 20A, a communication section 30A, and an input-output section 40A.

### (Communication Section 30A)

The communication section 30A communicates with an apparatus outside the information processing apparatus 1A via a communication line. A specific configuration of the communication line does not limit the present example embodiment, and the communication line is, for example, a wireless local area network (LAN), a wired LAN, a wide area network (WAN), a public network, a mobile data communication network, or a combination of these. The communication section 30A transmits data supplied from the control section 10A to another apparatus or supplies data received from another apparatus to the control section 10A.

### (Input-Output Section 40A)

Input-output apparatuses such as a keyboard, a mouse, a display, a printer, and a touch panel are connected to the input-output section 40A. The input-output section 40A receives input of various kinds of information with respect to the information processing apparatus 1A from a connected input apparatus. The input-output section 40A outputs various kinds of information to a connected output apparatus under control of the control section 10A. Examples of the input-output section 40A include an interface such as a universal serial bus (USB).

### (Control Section 10A)

As illustrated in FIG. 3, the control section 10A includes a generation phase execution section 100A, a training phase execution section 200A, and an inference phase execution section 300A. The generation phase execution section 100A executes a phase for generating training data. The training phase execution section 200A executes a training phase of a model M. The inference phase execution section 300A executes an inference phase using the model M.

The generation phase execution section 100A includes an acquisition section 11A, an extraction section 12A, a training data generation section 13A, and a second display section 17A. The training phase execution section 200A includes an evaluation model training section 18A. The inference phase execution section 300A includes an evaluation section 14A, an evaluation integration section 15A, and a first display section 16A.

### (Acquisition Section 11A)

The acquisition includes an section 11A electrocardiogram data acquisition section 111A and a division section 112A. The electrocardiogram data acquisition section 111A acquires electrocardiogram data D1. For example, the electrocardiogram data acquisition section 111A may acquire the electrocardiogram data D1 by retrieving the electrocardiogram data D1 from the storage section 20A or another external storage apparatus, or may acquire the electrocardiogram data D1 which is received from another apparatus via the communication section 30A. The acquisition section 11 may acquire electrocardiogram data D1 which is input through an input apparatus connected to the input-output section 40A.

The division section 112A divides a waveform indicated by the electrocardiogram data D1 into unit waveforms which are waveforms in predetermined cycles. For example, the division section 112A divides a waveform indicated by the electrocardiogram data D1 into unit waveforms by dividing the waveform indicated by the electrocardiogram data D1 by peak values (R waves). For example, the division section 112A may detect R waves from the waveform indicated by the electrocardiogram data D1 and detect a unit waveform corresponding to a section between a P wave and a T wave based on cycles identified from detection intervals of the R waves.

In the present example embodiment, the plurality of unit waveforms acquired by the acquisition section 11A are provided with labels. Examples of the label include a label indicating whether or not being a disease. More specifically, for example, a positive example label (e.g., value 1) is given to a unit waveform which is a positive example (disease), and a negative example label (e.g., value 0) is given to a unit waveform which is a negative example (normal). For example, the division section 112A provides, to each of unit waveforms after division, a label identical to a label attached to the electrocardiogram data D1 acquired by the electrocardiogram data acquisition section 111A.

### (Extraction Section 12A)

The extraction section 12A extracts, using a model M which has been trained with use of a certain group derived from the plurality of unit waveforms acquired by the acquisition section 11A, one or more unit waveforms from another group derived from the plurality of unit waveforms. The extraction section 12A includes a training section 121A. The training section 121A trains the model M using a certain group derived from the plurality of unit waveforms and labels which are provided to unit waveforms included in the certain group. The extraction section 12A extracts, using the model M, one or more unit waveforms from another group derived from the plurality of unit waveforms. A training process carried out by the training section 121A and an extraction process carried out by the extraction section 12A will be described later.

### (Training Data Generation Section 13A)

The training data generation section 13A generates training data including one or more unit waveforms which have been extracted by the extraction section 12A. The training data includes, for example, a set including data representing one or more unit waveforms and labels each indicating whether or not being a disease. The data representing one or more unit waveforms is, for example, data obtained by adding up a plurality of unit waveforms while aligning positions with peaks. Here, the process of adding up waveforms also includes a process of superimposing images of waveforms.

### (Second Display Section 17A)

The second display section 17A displays at least one of the plurality of unit waveforms which have been obtained by dividing the waveform indicated by the electrocardiogram data or at least one of the one or more unit waveforms which have been extracted by the extraction section 12A. The second display section 17A displays the unit waveform on a display connected to the input-output section 40A. A screen displayed on the second display section 17A is, for example, a screen for a doctor or a data scientist. The second display section 17A may receive, for example, (i) input of a positive example label or a negative example label, (ii) setting of a threshold (evaluation value threshold, or the like) which is referred to by the extraction section 12A, and the like, on the screen displayed on the second display section 17A. The (i) positive example label or negative example label is input by, for example, a doctor. The (ii) threshold referred to by the extraction section 12A is input by, for example, a doctor or a data scientist.

### (Evaluation Model Training Section 18A)

The evaluation model training section 18A trains an evaluation model LM using training data which has been generated by the training data generation section 13A, the evaluation model LM including the model M or being different from the model M. In a case where the evaluation model LM includes the model M, the evaluation model training section 18A retrains the model M using the training data. In a case where the evaluation model LM is different from the model M, the evaluation model training section 18A generates an evaluation model LM by supervised machine learning using the training data. A method of machine learning of the evaluation model LM is not limited. For example, a decision tree-based method, a linear regression method, or a neural network method may be used, or two or more of these methods may be used.

### (Evaluation Section 14A)

The evaluation section 14A evaluates, using the evaluation model LM which: has been trained by the evaluation model training section 18A, a plurality of unit waveforms obtained from evaluation electrocardiogram data for evaluation. For example, the evaluation section 14A receives evaluation electrocardiogram data from another apparatus connected via the communication section 30A, and inputs a plurality of unit waveforms obtained from the received evaluation electrocardiogram data into the evaluation model LM to evaluate the plurality of unit waveforms. More specifically, for example, the evaluation section 14A evaluates a unit waveform for which an evaluation value output by the evaluation model LM is equal to or greater than a predetermined threshold as a positive example (disease), and evaluates a unit waveform for which an evaluation value is less than the threshold as a negative example (normal). Note that the evaluation method by the evaluation section 14A is not limited to the example described above, and the evaluation section 14A may evaluate a unit waveform by another method.

### (Evaluation Integration Section 15A)

The evaluation integration section 15A evaluates the evaluation electrocardiogram data with reference to evaluation by the evaluation section 14A with respect to the plurality of unit waveforms. For example, the evaluation integration section 15A evaluates the evaluation electrocardiogram data as a disease in a case where one or more unit waveforms which have been evaluated as a disease by the evaluation section 14A include a unit waveform for which an evaluation value obtained by the evaluation section 14A is equal to or greater than a predetermined value, or in a case where the number of unit waveforms which have been evaluated as a disease by the evaluation section 14A is equal to or greater than a predetermined value.

The evaluation integration section 15A outputs an evaluation result. For example, the evaluation integration section 15A may output the evaluation result by transmitting the evaluation result to another apparatus via the communication section 30A, or may output the evaluation result to an output apparatus connected to the input-output section 40A. Here, examples of the output apparatus include a display, a printer, a projector, and a speaker. The evaluation integration section 15A may output the evaluation result by writing the evaluation result in the storage section 20A or an external storage apparatus.

### (First Display Section 16A)

The first display section 16A displays an evaluation result by the evaluation integration section 15A. The first display section 16A displays the evaluation result on a display connected to the input-output section 40A. The evaluation result displayed on the first display section 16A is confirmed by, for example, a doctor.

### (Storage Section 20A)

The storage section 20A stores electrocardiogram data D1 acquired by the electrocardiogram data acquisition section 111A and stores training data D2 generated by the training data generation section 13A. The storage section 20A also stores the model M and the evaluation model LM. Note that the state in which the storage section 20A stores the model M and the evaluation model LM means that the storage section 20A stores parameters defining the model M and parameters defining the evaluation model LM.

### (Model M)

The model M is a model for evaluating a disease from an electrocardiogram and is generated by supervised machine learning. An input into the model M is data representing one or more unit waveforms extracted from the electrocardiogram data D1, and is, for example, image data representing one or more unit waveforms. The data representing one or more unit waveforms is not limited to image data, and may be data in other forms. An output of the model M includes, for example, a label indicating whether or not being a disease or a reliability with respect to a disease. The reliability is, for example, a real number of 0 to 1. A larger value indicates that a possibility of being a disease is higher. The model M may be a single model or may include a plurality of models (e.g., models Mi (i∈[N]; N is a natural number of 2 or more)).

### (Evaluation Model LM)

The evaluation model LM is a model which is trained by the evaluation model training section 18A. The evaluation model LM is a model including the model M or a model different from the model M. The evaluation model LM may include a plurality of models (e.g., models Mi (i∈[N])).

An input into the evaluation model LM is, for example, data representing one or more unit waveforms extracted from evaluation electrocardiogram data. The data representing one or more unit waveforms is, for example, image data. The data representing one or more unit waveforms is not limited to image data, and may be data in other forms. An output of the evaluation model LM includes, for example, a label indicating whether or not being a disease or a reliability with respect to a disease. The reliability is, for example, a real number of 0 to 1. A larger value indicates that a possibility of being a disease is higher.

In a case where the evaluation model LM includes the model M, the evaluation model training section 18A may be identical to the training section 121A, or the evaluation model training section 18A and the training section 121A may train models using a common library.

### <Flow of Information Processing Method S1A>

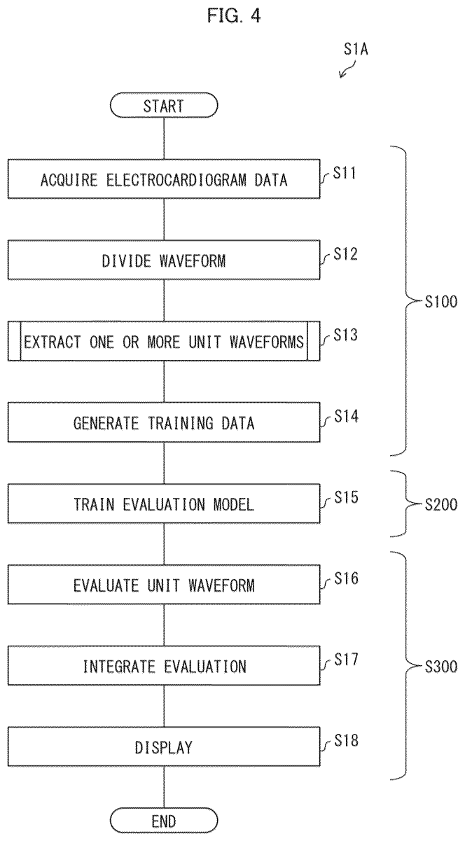

FIG. 4 is a flowchart illustrating a flow of an information processing method S1A which is an example of the information processing method carried out by the information processing apparatus 1A. Note that some of steps included in the information processing method S1A may be carried out in parallel or in a different order. Content which has already been described will not be repeated.

The information processing method S1A includes a data generation phase S100, a training phase S200, and an inference phase S300. The data generation phase S100 includes steps S11 through S14. The training phase S200 includes step S15. The inference phase S300 includes steps S16 through S18.

### (Step S11)

In step S11, the electrocardiogram data acquisition section 111A acquires electrocardiogram data D1. For example, the electrocardiogram data acquisition section 111A receives electrocardiogram data D1 from another apparatus connected via the communication section 30A. The electrocardiogram data D1 acquired by the electrocardiogram data acquisition section 111A is provided with a label indicating whether or not being a disease. For example, a positive example label (e.g., value 1) is given to electrocardiogram data D1 which is a positive example, and a negative example label (e.g., value 0) is given to electrocardiogram data D1 which is a negative example.

### (Step S12)

In step S12, the division section 112A divides a waveform indicated by the electrocardiogram data D1 into unit waveforms which are waveforms in predetermined cycles and provides a label to each of the unit waveforms which have been obtained by the division. For example, the division section 112A gives a positive example label (e.g., value 1) to each of unit waveforms included in electrocardiogram data D1 which is a positive example, and gives a negative example label (e.g., value 0) to each of unit waveforms included in electrocardiogram data D1 which is a negative example.

### (Step S13)

In step S13, the extraction section 12A extracts, using the model M which has been trained with use of a certain group derived from the plurality of divided unit waveforms, one or more unit waveforms from another group derived from the plurality of unit waveforms. Details of the extraction process carried out by the extraction section 12A will be described later.

Values of parameters (evaluation value threshold, and the like) related to the extraction process by the extraction section 12A may each be a predetermined value or may each be a value designated by a user. In a case where the user designates a parameter, for example, the second display section 17A displays a screen for designating the parameter on the display apparatus, and the extraction section 12A sets a threshold of the evaluation value or the like based on information input by the user on the displayed screen.

### (Step S14)

In step S14, the training data generation section 13A generates training data D2 including one or more unit waveforms which have been extracted by the extraction section 12A. For example, the training data generation section 13A may generate an added unit waveform by adding up a plurality of unit waveforms extracted by the extraction section 12A, and generate training data D2 which includes the added unit waveform which has been generated. Here, addition of unit waveforms also includes superposition of images representing waveforms.

### (Step S15)

In step S15, the evaluation model training section 18A trains the evaluation model LM using the training data D2 which has been generated by the training data generation section 13A. In a case where the evaluation model LM includes the model M, the evaluation model training section 18A retrains the model M using the training data D2. In a case where the evaluation model LM is different from the model M, the evaluation model training section 18A trains the evaluation model LM by supervised machine learning using the training data D2.

### (Step S16)

In step S16, the evaluation section 14A evaluates, using the evaluation model LM which has been trained by the evaluation model training section 18A, a plurality of unit waveforms obtained from evaluation electrocardiogram data. The evaluation section 14A carries out, for example, (i) acquisition of evaluation electrocardiogram data, (ii) decomposition into unit waveforms, and (iii) input of unit waveforms into the evaluation model LM.

In this case, the evaluation section 14A first acquires evaluation electrocardiogram data. For example, the evaluation section 14A receives evaluation electrocardiogram data from another apparatus connected via the communication section 30A. Next, the evaluation section 14A divides the evaluation electrocardiogram data into a plurality of unit waveforms. The division method is similar to the division method carried out by the division section 112A. The evaluation section 14A inputs a plurality of unit waveforms obtained by division into the evaluation model LM to evaluate the plurality of unit waveforms. Examples of the unit waveforms which the evaluation section 14A inputs into the evaluation model LM include:
any of a plurality of unit waveforms obtained by dividing evaluation electrocardiogram data;
a unit waveform extracted by the extraction section 12A from a plurality of unit waveforms which have been obtained by dividing evaluation electrocardiogram data; and
an added unit waveform obtained by adding up unit waveforms extracted by the extraction section 12A.

For example, the evaluation section 14A evaluates a unit waveform for which an evaluation value output by the evaluation model LM is equal to or greater than a threshold Th1 as a positive example (disease), and evaluates a unit waveform for which the evaluation value is less than the threshold Th1 as a negative example (normal).

In a case where the evaluation model LM includes a plurality of models (e.g., models M1, M2, . . . . MN), the evaluation section 14A may evaluate a unit waveform by integrating outputs which have been obtained by inputting a single unit waveform into the plurality of models. In this case, for example, the evaluation section 14A may evaluate a unit waveform using an average value or a weighted average of a plurality of evaluation values. The evaluation section 14A may evaluate a unit waveform based on the number of positive example labels and the number of negative example labels output by the plurality of models. For example, in a case where the number of positive example labels is greater than the number of negative example labels, the unit waveform may be evaluated as a positive example.

### (Step S17)

In step S17, the evaluation integration section 15A evaluates the evaluation electrocardiogram data with reference to evaluation by the evaluation section 14A with respect to the plurality of unit waveforms. For example, the evaluation integration section 15A evaluates, as a disease, evaluation electrocardiogram data in which the number of unit waveforms which have been evaluated as a disease in step S16 is equal to or greater than a predetermined threshold. For example, the evaluation integration section 15A may evaluate, as a disease, evaluation electrocardiogram data including one or more unit waveforms which are equal to or greater than a threshold Th2 (Th2>Th1) from among pieces of evaluation electrocardiogram data including unit waveforms which have been evaluated as a disease in step S16.

### (Step S18)

The first display section 16A displays an evaluation result by the evaluation integration section 15A. For example, the first display section 16A displays information indicating whether or not an evaluation result of evaluation electrocardiogram data is a disease.

### <Descriptions of Process of Extracting Unit Waveform by Extraction Section 12A>

The following description will discuss process examples 1 through 5 as specific examples of the extraction process (step S13 in FIG. 4) which is carried out by the extraction section 12A.

### Process Example 1

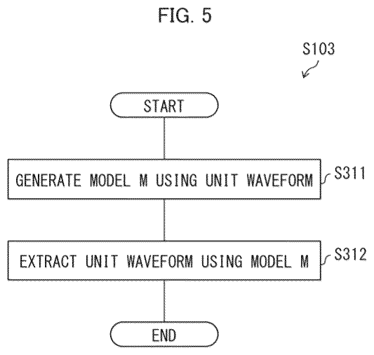

FIG. 5 is a flowchart illustrating a flow of process example 1. In step S311, the extraction section 12A generates a model M by supervised machine learning in which unit waveforms obtained by dividing electrocardiogram data D1 are used as training data. Here, the training data used by the extraction section 12A for machine learning of the model M includes a plurality of sets of unit waveforms which have been obtained by division by the division section 112A and labels. A method of machine learning of the model M is not limited. For example, a decision tree-based method, a linear regression method, or a neural network method may be used, or two or more of these methods may be used.

In step S312, the extraction section 12A extracts one or more unit waveforms by evaluating, using the model M generated in step S311, at least a part of the plurality of unit waveforms which have been obtained by dividing the electrocardiogram data D1. More specifically, the extraction section 12A inputs at least a part of the plurality of unit waveforms obtained by dividing the electrocardiogram data D1 into the model M, and extracts unit waveforms which are true positive (TP) and true negative (TN) based on outputs of the model M.

### Process Example 2

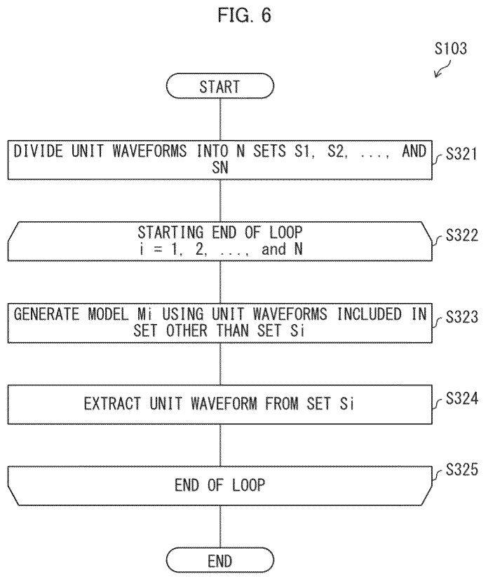

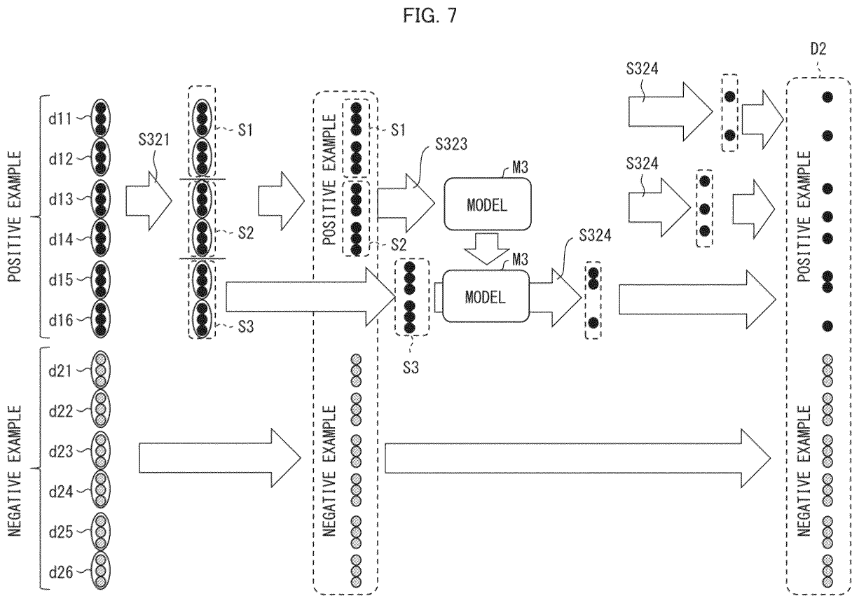

FIG. 6 is a flowchart illustrating a flow of an extraction process in process example 2. FIG. 7 is a diagram exemplifying unit waveforms extracted in process example 2. In process example 2, the extraction section 12A extracts unit waveforms using N (N is a natural number of 2 or more) models Mi (i∈[N]).

### (Step S321)

In step S321, the training section 121A divides the plurality of unit waveforms acquired by the acquisition section 11A into N sets. In other words, the extraction section 12A divides, into N sets S1, S2, . . . , and SN, the plurality of unit waveforms obtained by dividing the electrocardiogram data D1. Here, each of the sets Si (i∈[N]) includes a plurality of unit waveforms. For example, the extraction section 12A groups the plurality of unit waveforms included in the electrocardiogram data D1 for each predetermined number. A method in which the extraction section 12A divides the plurality of unit waveforms into N sets is not limited to the example described above, and the extraction section 12A may divide the plurality of unit waveforms into N sets by another method.

In the example illustrated in FIG. 7, pieces of electrocardiogram data d11 through d16 which are positive examples and pieces of electrocardiogram data d21 through d26 which are negative examples each include unit waveforms for three cycles. In step S321, the extraction section 12A divides a plurality of unit waveforms included in the pieces of electrocardiogram data d11 through d16 into sets S1 through S3.

### (Step S322)

Step S322 is a starting end of a loop process related to a model Mi. Here, a loop variable i is a natural number satisfying 1≤i≤N.

### (Step S323)

In step S323, the training section 121A trains each of the plurality of models Mi using each of a plurality of groups of the plurality of unit waveforms acquired by the acquisition section 11A. Here, the plurality of groups include a set(s) other than a set Si. In other words, the training section 121A generates a model Mi by supervised machine learning in which unit waveforms included in a set(s) other than the set Si are used as training data. A method of machine learning of the model Mi is not limited. For example, a decision tree-based method, a linear regression method, or a neural network method may be used, or two or more of these methods may be used.

In the example of FIG. 7, in a case where i=3, the extraction section 12A generates a model M3 by supervised machine learning using, as training data, unit waveforms included in a set S1, unit waveforms included in a set S2, and unit waveforms included in the pieces of electrocardiogram data d21 through d26 which are negative examples.

In the example of FIG. 7, the extraction section 12A extracts all unit waveforms included in the pieces of electrocardiogram data d21 through d26 which are negative examples. A method in which the extraction section 12A extracts unit waveforms from negative examples is not limited to the example described above, and the extraction section 12A may extract unit waveforms included in electrocardiogram data which is a negative example by sampling at a predetermined rate.

### (Step S324)

In step S324, the extraction section 12A extracts, using each of the models Mi, one or more unit waveforms from another group different from a group used for training that model Mi. Here, another group different from the group used for the training of the model Mi is unit waveforms included in the set Si. In other words, the extraction section 12A extracts one or more unit waveforms from the set Si by evaluating, using the model Mi generated in step S323, unit waveforms included in the set Si.

More specifically, the extraction section 12A extracts a unit waveform for which an evaluation value obtained by the model Mi is equal to or greater than a predetermined threshold from among a plurality of unit waveforms included in the foregoing another group. In other words, the extraction section 12A inputs unit waveforms included in the set Si into the model Mi and extracts a unit waveform for which an evaluation value output by the model Mi is equal to or greater than a predetermined threshold. A method in which the extraction section 12A extracts one or more unit waveforms from the set Si is not limited to the example described above, and the extraction section 12A may extract one or more unit waveforms from the set Si by another method based on an output of the model Mi.

In the example of FIG. 7, when i=3, the extraction section 12A inputs unit waveforms included in the set S3 into the model M3, and extracts a plurality of unit waveforms from the set S3 based on outputs of the model M3.

### (Step S325)

Step S325 is the end of the loop process related to the loop variable i.

In process example 2, the extraction section 12A carries out the process illustrated in FIG. 6 to extract unit waveforms from each of the sets Si (i∈[N]). The training data generation section 13A generates training data D2 including unit waveforms which have been extracted by the extraction section 12A. In the example of FIG. 7, the training data generation section 13A generates training data D2 including unit waveforms which have been extracted by the extraction section 12A from each of the sets S1 through S3 and unit waveforms which are included in the pieces of electrocardiogram data d21 through d26 which are negative examples.

Thus, in steps S322 through S325, in process example 2, the training section 121A trains each of the N models Mi using each of N groups each of which excludes one set in turn from the N sets 1 through SN. Here, each of the N groups includes (N−1) sets. The extraction section 12A extracts, using each of the N models Mi, one or more unit waveforms from one set which has not been used to train that model Mi.

### Process Example 3

In step S324 in process example 2, the extraction section 12A inputs unit waveforms included in the set Si into the model Mi and extracts a unit waveform for which an evaluation value output by the model Mi is equal to or greater than a predetermined threshold. Meanwhile, in process example 3, the extraction section 12A extracts, in step S324, a unit waveform for which an evaluation value obtained by the model Mi is greatest from among a plurality of unit waveforms included in the foregoing another group. More specifically, for example, the extraction section 12A extracts, from each of pieces of electrocardiogram data D1 included in the sets Si, a unit waveform for which an evaluation value output by the model Mi is highest. That is, the extraction section 12A extracts one unit waveform from each of pieces of electrocardiogram data D1 which are positive examples. In this case, the training data generation section 13A generates training data D2 including a unit waveform which has been extracted by the extraction section 12A and unit waveforms which are included in electrocardiogram data D1 which is a negative example.

For the negative example, the extraction section 12A may extract all unit waveforms included in the electrocardiogram data D1 which is a negative example, or may extract unit waveforms included in the electrocardiogram data D1 which is a negative example by sampling at a predetermined rate.

### Process Example 4

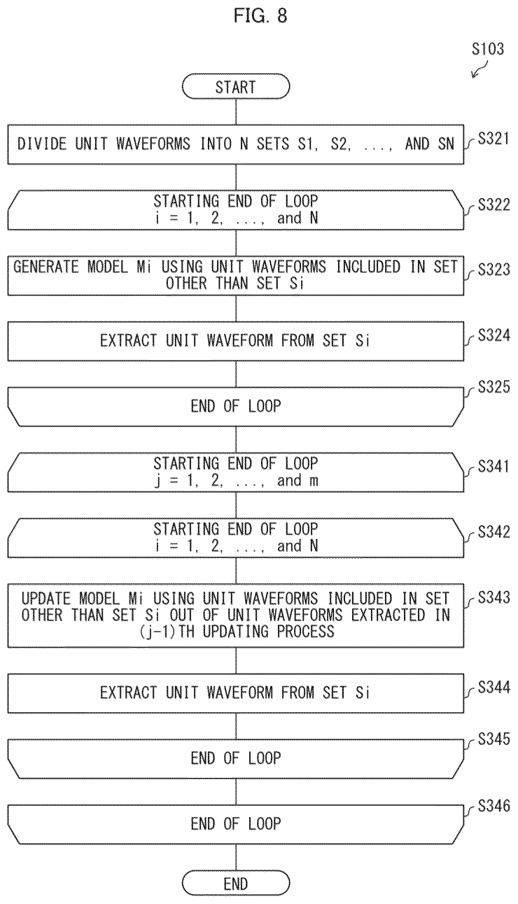

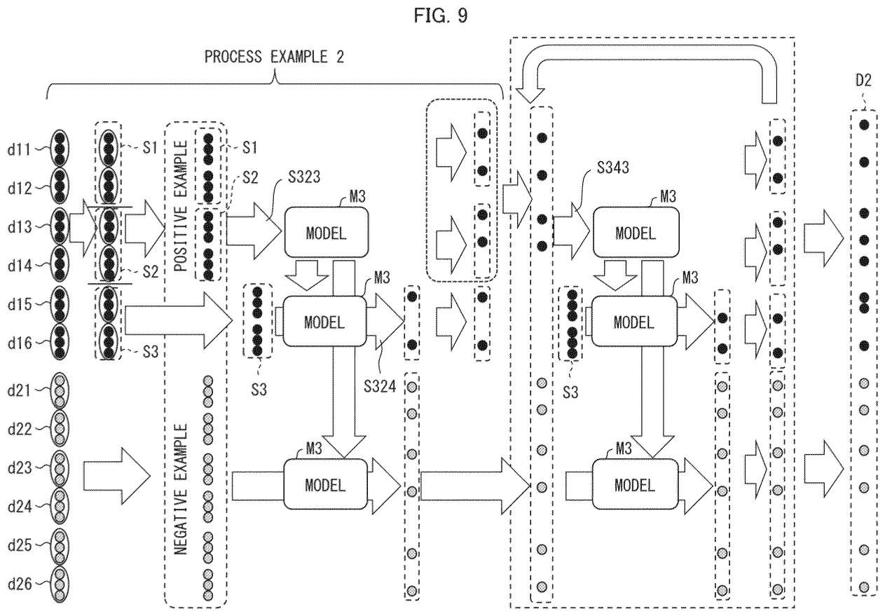

FIG. 8 is a flowchart illustrating a flow of process example 4. FIG. 9 is a diagram exemplifying unit waveforms extracted in process example 4. In process example 4, the extraction section 12A repeatedly carries out an updating process of the model Mi generated in process example 2 and an evaluation process using the updated model Mi.

In process example 4, the process carried out by the extraction section 12A includes steps S321 through S325 in process example 2 and steps S341 through S345 subsequent to step S325. The processes of steps S321 through S325 have already been described in process example 2 above, and therefore the descriptions thereof will not be repeated here.

### (Step S341)

Step S341 is a starting end of a loop process related to a process of updating a model Mi. Here, a loop variable j in the loop process related to the updating process is a natural number satisfying 1≤j≤m. m is the number of times of update of the model Mi, and is a natural number of 1 or more.

### (Step S342 Through Step S345)

In steps S342 through S345, the extraction section 12A updates any of the models Mi using a certain group derived from the plurality of unit waveforms which have been extracted using each of the models Mi, and the extraction section 12A extracts, using the updated model Mi, one or more unit waveforms from another group derived from the plurality of unit waveforms which have been extracted using each of the models Mi.

### (Step S342)

Step S342 is a starting end of a loop process related to the model Mi. Here, a loop variable i is a natural number satisfying 1≤i≤N.

### (Step S343)

In step S343, the extraction section 12A updates the model Mi using unit waveforms included in a set(s) other than the set Si out of unit waveforms extracted in a (j−1)th updating process. That is, the extraction section 12A retrains the model Mi using, as training data, unit waveforms which have been extracted in the (j−1)th updating process and are not included in the set Si. Note, however, that, in a case where j=1, the extraction section 12A uses, as training data, unit waveforms extracted in step S324. In the example of FIG. 9, in step S343, the extraction section 12A updates the model M3 while using, as training data, unit waveforms which are not included in the set S3 out of unit waveforms which have been extracted in the (j−1)th update (or unit waveforms extracted in step S324).

### (Step S344)

In step S344, the extraction section 12A extracts one or more unit waveforms from the set Si by evaluating, using the model Mi updated in step S343, unit waveforms included in the set Si. The process of step S344 is similar to the process of step S324 described above.

### (Step S345)

Step S345 is the end of the loop process related to the loop variable i.

### (Step S346)

Step S346 is the end of the loop process related to the loop variable j.

Thus, in process example 4, in steps S341 through S346, the extraction section 12A carries out updating of a model Mi and extraction using the model Mi two or more times while altering a certain group derived from the plurality of unit waveforms which have been extracted using each of the models Mi.

In process example 4, the training data generation section 13A generates training data D2 including unit waveforms which have been extracted in m-th step S344 by the extraction section 12A.

### Process Example 5

In process example 5, the extraction section 12A repeatedly carries out an updating process of the model Mi generated in process example 3 and an evaluation process using the updated model Mi. More specifically, in process example 5, in step S344 of FIGS. 8 and 9 described in process example 4, the extraction section 12A extracts one unit waveform for which an evaluation value is highest from each of a plurality of pieces of electrocardiogram data D1.

### <Process Example Carried Out by Training Data Generation Section 13A>

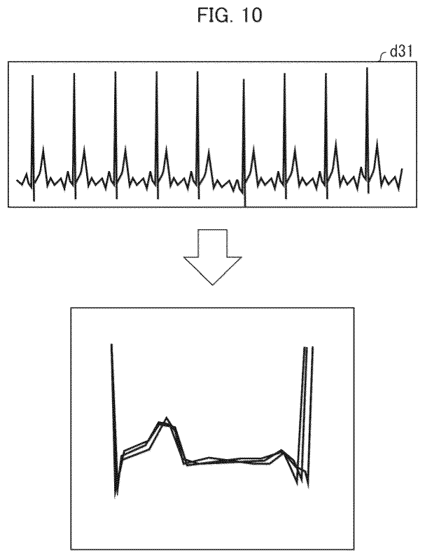

FIG. 10 is a diagram illustrating an example of a training data generation process carried out by the training data generation section 13A. In the example of FIG. 10, electrocardiogram data d31 includes a plurality of unit waveforms. In the example of FIG. 10, the training data generation section 13A generates an image in which one or more unit waveform images extracted by the extraction section 12A from the plurality of unit waveforms included in the electrocardiogram data d31 are superimposed while aligning positions at peaks.

### Display Example 1

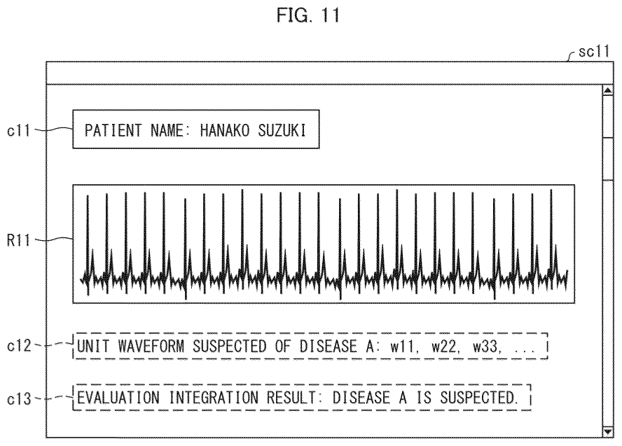

FIG. 11 is a diagram illustrating a display screen example which is displayed on the first display section 16A. In the example illustrated in FIG. 11, the first display section 16A displays an evaluation result by the evaluation integration section 15A. A screen sc11 includes an electrocardiogram R11, identification information c11, an evaluation result c12, and an evaluation integration result c13. The electrocardiogram R11 is an electrocardiogram indicated by evaluation electrocardiogram data. The identification information c11 is information identifying the electrocardiogram R11 and includes, for example, a patient name. The evaluation result c12 is an evaluation result by the evaluation section 14A and includes, for example, a list of unit waveforms which have been evaluated as a disease. The evaluation integration result c13 includes information indicating an evaluation result by the evaluation integration section 15A. A user can ascertain an evaluation result of a disease through the screen displayed on the first display section 16A.

### Display Example 2

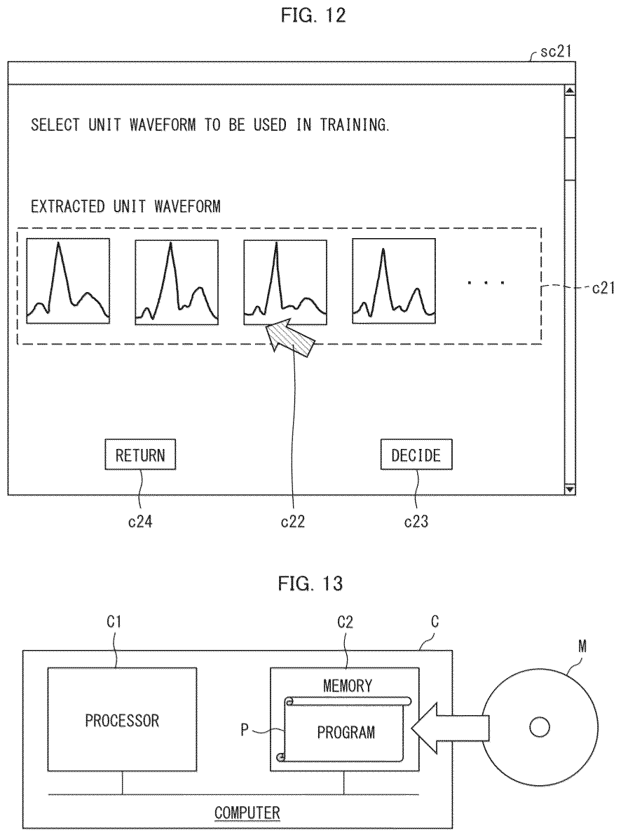

FIG. 12 is a diagram illustrating a display screen example which is displayed on the second display section 17A. In the example of FIG. 12, the second display section 17A displays a unit waveform which has been extracted by the extraction section 12A. The screen sc21 includes a unit waveform group c21, a pointer c22, a decision button c23, and a button c24. The unit waveform group c21 is a set of unit waveforms extracted by the extraction section 12A. The pointer c22 is a pointer for selecting a unit waveform from the unit waveform group c21. The user selects a unit waveform by carrying out operation such as moving the pointer c22 using an input apparatus connected to the input-output section 40A. The decision button c23 is a button for deciding a unit waveform to be selected. The button c24 is a button for ending selection of a unit waveform.

The screen displayed on the second display section 17A is not limited to the example illustrated in FIG. 12, and may be another screen. For example, the second display section 17A may display a screen for setting a parameter such as a threshold related to extraction of a unit waveform. In this case, the user sets a parameter such as a threshold using an input apparatus connected to the input-output section 40A. The extraction section 12A extracts a unit waveform based on the set threshold.

### <Example Advantage of Information Processing Apparatus 1A>

As described above, the information processing apparatus 1A in accordance with the present example embodiment employs the configuration in which the acquisition section 11A includes: the electrocardiogram data acquisition section 111A for acquiring electrocardiogram data D1; and the division section 112A for dividing a waveform indicated by the electrocardiogram data D1 into unit waveforms which are waveforms in predetermined cycles. Instead of generating training data using all pieces of electrocardiogram data D1, training data D2 is generated using a unit waveform which has been extracted using the model M. This makes it possible to bring about an example advantage of heightening accuracy of the training data D2.

The information processing apparatus 1A in accordance with the present example embodiment employs the configuration in which: the plurality of unit waveforms acquired by the acquisition section 11A are provided with labels; and the extraction section 12A includes the training section 121A for training the model M with use of a certain group derived from the plurality of unit waveforms and labels which are provided to unit waveforms included in the certain group. By training the model M using a part of a plurality of unit waveforms and extracting unit waveforms for generating training data using the model M which has been trained, the information processing apparatus 1A in accordance with the present example embodiment makes it possible to bring about an example advantage of further heightening accuracy of the training data D2 which is generated.

The information processing apparatus 1A in accordance with the present example embodiment employs the configuration in which: the training section 121A trains a plurality of models Mi using a plurality of groups, respectively, which are derived from the plurality of unit waveforms acquired by the acquisition section 11A; and the extraction section 12A extracts, using each of the models Mi, one or more unit waveforms from another group different from a group which has been used to train that model Mi. Therefore, according to the information processing apparatus 1A in accordance with the present example embodiment, it is possible to bring about an example advantage of further heightening accuracy of the training data D2 which is generated.

The information processing apparatus 1A in accordance with the present example embodiment employs the configuration in which: the training section 121A divides the plurality of unit waveforms acquired by the acquisition section 11A into N sets, and trains each of N models Mi using each of N groups each of which excludes one set in turn from the N sets; and the extraction section 12A extracts, using each of the N models Mi, one or more unit waveforms from one set which has not been used to train that model Mi. Therefore, according to the information processing apparatus 1A in accordance with the present example embodiment, it is possible to bring about an example advantage of further heightening accuracy of the training data D2.

The information processing apparatus 1A in accordance with the present example embodiment employs the configuration in which: the extraction section 12A updates any of the models Mi using a certain group derived from the plurality of unit waveforms which have been extracted using each of the models Mi, and the extraction section 12A extracts, using the updated model Mi, one or more unit waveforms from another group derived from the plurality of unit waveforms which have been extracted using each of the models. By updating any of the models Mi using unit waveforms extracted by the extraction section 12A and extracting unit waveforms for generating training data using the updated model Mi, the information processing apparatus 1A in accordance with the present example embodiment brings about an example advantage of further heightening accuracy of the training data D2.

The information processing apparatus 1A in accordance with the present example embodiment employs the configuration in which: the extraction section 12A carries out updating of a model Mi and extraction using the model Mi two or more times while altering a certain group derived from the plurality of unit waveforms which have been extracted using each of the models Mi. By repeating the extraction process using the model Mi, the information processing apparatus 1A in accordance with the present example embodiment makes it possible to bring about an example advantage of further heightening accuracy of the training data D2.

The information processing apparatus 1A in accordance with the present example embodiment employs the configuration in which: the extraction section 12A extracts a unit waveform for which an evaluation value obtained by the model M is equal to or greater than a predetermined threshold from among a plurality of unit waveforms included in the foregoing another group. Therefore, according to the information processing apparatus 1A in accordance with the present example embodiment, it is possible to bring about an example advantage of generating training data D2 for evaluating a disease from an electrocardiogram with higher accuracy.

The information processing apparatus 1A in accordance with the present example embodiment employs the configuration in which: the extraction section 12A extracts a unit waveform for which an evaluation value obtained by the model M is greatest from among a plurality of unit waveforms included in the foregoing another group. Therefore, according to the information processing apparatus 1A in accordance with the present example embodiment, it is possible to bring about an example advantage of generating training data D2 for evaluating a disease from an electrocardiogram with higher accuracy.

The information processing apparatus 1A in accordance with the present example embodiment employs the configuration in which: the training data generation section 13A generates an added unit waveform by adding up a plurality of unit waveforms extracted by the extraction section 12A, and generates the training data D2 which includes the added unit waveform which has been generated. Therefore, according to the information processing apparatus 1A in accordance with the present example embodiment, it is possible to bring about an example advantage of generating training data D2 for evaluating a disease from an electrocardiogram with higher accuracy.

The information processing apparatus 1A in accordance with the present example embodiment employs the configuration of further including: the evaluation model training section 18A for training an evaluation model LM using the training data D2 which has been generated by the training data generation section 13A, the evaluation model LM including the model M or being different from the model M. Therefore, according to the information processing apparatus 1A in accordance with the present example embodiment, in addition to the example advantage brought about by the information processing apparatus 1 in accordance with the first example embodiment, it is possible to bring about an example advantage of generating an evaluation model LM which can evaluate a disease from an electrocardiogram with higher accuracy.

The information processing apparatus 1A in accordance with the present example embodiment employs the configuration of further including: the evaluation section 14A for evaluating, using the evaluation model LM which has been trained by the evaluation model training section 18A, a plurality of unit waveforms that are obtained from evaluation electrocardiogram data for evaluation; and the evaluation integration section 15A for evaluating the evaluation electrocardiogram data with reference to evaluation by the evaluation section 14A with respect to the plurality of unit waveforms. Therefore, according to the information processing apparatus 1A in accordance with the present example embodiment, in addition to the example advantage brought about by the information processing apparatus 1 in accordance with the first example embodiment, it is possible to bring about an example advantage of evaluating a disease from an electrocardiogram with higher accuracy.

The information processing apparatus 1A in accordance with the present example embodiment employs the configuration in which: the evaluation integration section 15A evaluates the evaluation electrocardiogram data as a disease in a case where one or more unit waveforms which have been evaluated as a disease by the evaluation section 14A include a unit waveform for which an evaluation value obtained by the evaluation section 14A is equal to or greater than a predetermined value, or in a case where the number of unit waveforms which have been evaluated as a disease by the evaluation section 14A is equal to or greater than a predetermined value. Therefore, according to the information processing apparatus 1A in accordance with the present example embodiment, in addition to the example advantage brought about by the information processing apparatus 1 in accordance with the first example embodiment, it is possible to bring about an example advantage of evaluating a disease from an electrocardiogram with higher accuracy.

The information processing apparatus 1A in accordance with the present example embodiment employs the configuration of further including: the first display section 16A for displaying an evaluation result by the evaluation integration section 15A. Therefore, according to the information processing apparatus 1A in accordance with the present example embodiment, in addition to the example advantage brought about by the information processing apparatus 1 in accordance with the first example embodiment, it is possible to bring about an example advantage of allowing a user to ascertain an evaluation result.

The information processing apparatus 1A in accordance with the present example embodiment employs the configuration of further including: the second display section 17A for displaying at least one of the plurality of unit waveforms which have been obtained by dividing the waveform indicated by the electrocardiogram data or at least one of the one or more unit waveforms which have been extracted by the extraction section 12A. Therefore, according to the information processing apparatus 1A in accordance with the present example embodiment, in addition to the example advantage brought about by the information processing apparatus 1 in accordance with the first example embodiment, it is possible to bring about an example advantage of allowing a user to ascertain at least one of the plurality of unit waveforms which have been obtained by dividing the waveform indicated by the electrocardiogram data or at least one of the one or more unit waveforms which have been extracted by the extraction section 12A.

### Software Implementation Example

Some or all of the functions of each of the information processing apparatuses 1 and 1A may be implemented by hardware such as an integrated circuit (IC chip), or may be implemented by software.

In the latter case, the information processing apparatuses 1 and 1A are implemented by, for example, a computer that executes instructions of a program that is software implementing the foregoing functions. FIG. 13 illustrates an example of such a computer (hereinafter, referred to as “computer C”). The computer C includes at least one processor C1 and at least one memory C2. The memory C2 stores a program P for causing the computer C to operate as the information processing apparatuses 1 and 1A. The processor C1 of the computer C retrieves the program P from the memory C2 and executes the program P, so that the functions of the information processing apparatuses 1 and 1A are implemented.

Examples of the processor C1 include a central processing unit (CPU), a graphic processing unit (GPU), a digital signal processor (DSP), a micro processing unit (MPU), a floating point number processing unit (FPU), a physics processing unit (PPU), a microcontroller, and a combination thereof. Examples of the memory C2 include a flash memory, a hard disk drive (HDD), a solid state drive (SSD), and a combination thereof.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a computer C-readable, non-transitory, and tangible storage medium M. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communication network, a broadcast wave, or the like. The computer C can obtain the program P also via such a transmission medium.

### [Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

### [Additional Remark 2]

Some or all of the foregoing example embodiments can also be described as below. Note, however, that the present invention is not limited to the following supplementary notes.

### (Supplementary Note 1)

An information processing apparatus, including: an acquisition means for acquiring a plurality of unit waveforms which have been obtained by dividing a waveform indicated by electrocardiogram data; an extraction means for extracting, using a model which has been trained with use of a certain group derived from the plurality of unit waveforms acquired by the acquisition means, one or more unit waveforms from another group derived from the plurality of unit waveforms; and a training data generation means for generating training data including the one or more unit waveforms which have been extracted by the extraction means.

### (Supplementary Note 2)

The information processing apparatus according to supplementary note 1, in which the acquisition means includes: an electrocardiogram data acquisition means for acquiring the electrocardiogram data; and a division means for dividing the waveform indicated by the electrocardiogram data into unit waveforms which are waveforms in predetermined cycles.

### (Supplementary Note 3)

The information processing apparatus according to supplementary note 1 or 2, in which: the plurality of unit waveforms acquired by the acquisition means are provided with labels; and the extraction means includes a training means for training the model with use of a certain group derived from the plurality of unit waveforms and labels which are provided to unit waveforms included in the certain group.

### (Supplementary Note 4)

The information processing apparatus according to supplementary note 3, in which: the training means trains a plurality of models using a plurality of groups, respectively, which are derived from the plurality of unit waveforms acquired by the acquisition means; and the extraction means extracts, using each of the plurality of models, one or more unit waveforms from another group different from a group which has been used to train that model.

### (Supplementary Note 5)

The information processing apparatus according to supplementary note 4, in which: the training means divides the plurality of unit waveforms acquired by the acquisition means into N sets, and trains each of N models using each of N groups each of which excludes one set in turn from the N sets; and the extraction means extracts, using each of the N models, one or more unit waveforms from one set which has not been used to train that model.

### (Supplementary Note 6)

The information processing apparatus according to supplementary note 4 or 5, in which: the extraction means updates any of the plurality of models using a certain group derived from the plurality of unit waveforms which have been extracted using each of the plurality of models; and the extraction means extracts, using a model which has been updated, one or more unit waveforms from another group derived from the plurality of unit waveforms which have been extracted using each of the plurality of models.

### (Supplementary Note 7)

The information processing apparatus according to supplementary note 6, in which: the extraction means carries out updating of a model and extraction using the model two or more times while altering a certain group derived from the plurality of unit waveforms which have been extracted using each of the plurality of models.

### (Supplementary Note 8)

The information processing apparatus according to any one of supplementary notes 1 through 7, in which: the extraction means extracts a unit waveform for which an evaluation value obtained by the model is equal to or greater than a predetermined threshold from among a plurality of unit waveforms included in the another group.

### (Supplementary Note 9)

The information processing apparatus according to any one of supplementary notes 1 through 7, in which: the extraction means extracts a unit waveform for which an evaluation value obtained by the model is greatest from among a plurality of unit waveforms included in the another group.

### (Supplementary Note 10)

The information processing apparatus according to any one of supplementary notes 1 through 9, in which: the training data generation means generates an added unit waveform by adding up a plurality of unit waveforms extracted by the extraction means, and generates the training data including the added unit waveform which has been generated.

### (Supplementary Note 11)

The information processing apparatus according to any one of supplementary notes 1 through 10, further including: an evaluation model training means for training an evaluation model using the training data which has been generated by the training data generation means, the evaluation model including the model or being different from the model.

### (Supplementary Note 12)

The information processing apparatus according to supplementary note 11, further including: an evaluation means for evaluating, using the evaluation model which has been trained by the evaluation model training means, a plurality of unit waveforms that are obtained from evaluation electrocardiogram data for evaluation; and an evaluation integration means for evaluating the evaluation electrocardiogram data with reference to evaluation by the evaluation means with respect to the plurality of unit waveforms.

### (Supplementary Note 13)

The information processing apparatus according to supplementary note 12, in which: the evaluation integration means evaluates the electrocardiogram data as a disease in a case where one or more unit waveforms which have been evaluated as a disease by the evaluation means include a unit waveform for which an evaluation value obtained by the evaluation means is equal to or greater than a predetermined value, or in a case where the number of unit waveforms which have been evaluated as a disease by the evaluation means is equal to or greater than a predetermined value.

### (Supplementary Note 14)

The information processing apparatus according to supplementary note 13, further including: a first display means for displaying an evaluation result by the evaluation integration means.

### (Supplementary Note 15)

The information processing apparatus according to any one of supplementary notes 1 through 14, further including: a second display means for displaying at least one of the plurality of unit waveforms which have been obtained by dividing the waveform indicated by the electrocardiogram data or at least one of the one or more unit waveforms which have been extracted by the extraction means.

### (Supplementary Note 16)

An information processing method, including: acquiring, by at least one processor, a plurality of unit waveforms which have been obtained by dividing a waveform indicated by electrocardiogram data; extracting, by the at least one processor, using a model which has been trained with use of a certain group derived from the plurality of unit waveforms, one or more unit waveforms from another group derived from the plurality of unit waveforms; and generating, by the at least one processor, training data including the one or more unit waveforms which have been extracted.

### (Supplementary Note 17)

An information processing program for causing a computer to carry out: an acquisition process of acquiring a plurality of unit waveforms which have been obtained by dividing a waveform indicated by electrocardiogram data; an extraction process of extracting, using a model which has been trained with use of a certain group derived from the plurality of unit waveforms acquired in the acquisition process, one or more unit waveforms from another group derived from the plurality of unit waveforms; and a training data generation process of generating training data including the one or more unit waveforms which have been extracted in the extraction process.

### [Additional Remark 3]

Furthermore, some of or all of the foregoing example embodiments can also be expressed as below.

An information processing apparatus including at least one processor, the at least one processor carrying out: an acquisition process of acquiring a plurality of unit waveforms which have been obtained by dividing a waveform indicated by electrocardiogram data; an extraction process of extracting, using a model which has been trained with use of a certain group derived from the plurality of unit waveforms acquired in the acquisition process, one or more unit waveforms from another group derived from the plurality of unit waveforms; and a training data generation process of generating training data including the one or more unit waveforms which have been extracted in the extraction process.

Note that the information processing apparatus can further include a memory. The memory can store a program for causing the at least one processor to carry out the acquisition process, the extraction process, and the training data generation process. The program can be stored in a computer-readable non-transitory tangible storage medium.

### REFERENCE SIGNS LIST

1, 1A: Information processing apparatus
11, 11A: Acquisition section
12, 12A: Extraction section
13, 13A: Training data generation section
14A: Evaluation section
15A: Evaluation integration section
16A: First display section
17A: Second display section
18A: Evaluation model training section
111A: Electrocardiogram data acquisition section
S100, S1A: Information processing method

What is claimed is:

1. An information processing apparatus, comprising at least one processor, the at least one processor carrying out:
an acquisition process of acquiring a plurality of unit waveforms which have been obtained by dividing a waveform indicated by electrocardiogram data;
an extraction process of extracting, using a model which has been trained with use of a certain group derived from the plurality of unit waveforms acquired in the acquisition process, one or more unit waveforms from another group derived from the plurality of unit waveforms; and
a training data generation process of generating training data including the one or more unit waveforms which have been extracted in the extraction process.

2. The information processing apparatus according to claim 1, wherein, in the acquisition process, the at least one processor carries out:
an electrocardiogram data acquisition process of acquiring the electrocardiogram data; and
a division process of dividing the waveform indicated by the electrocardiogram data into unit waveforms which are waveforms in predetermined cycles.

3. The information processing apparatus according to claim 1, wherein:
the plurality of unit waveforms acquired by the at least one processor in the acquisition process are provided with labels; and
in the extraction process, the at least one processor carries out a training process of training the model with use of a certain group derived from the plurality of unit waveforms and labels which are provided to unit waveforms included in the certain group.

4. The information processing apparatus according to claim 3, wherein:
in the training process, the at least one processor trains a plurality of models using a plurality of groups, respectively, which are derived from the plurality of unit waveforms acquired in the acquisition process; and
in the extraction process, the at least one processor extracts, using each of the plurality of models, one or more unit waveforms from another group different from a group which has been used to train that model.

5. The information processing apparatus according to claim 4, wherein:
in the training process, the at least one processor divides the plurality of unit waveforms acquired in the acquisition process into N sets, and trains each of N models using each of N groups each of which excludes one set in turn from the N sets; and
in the extraction process, the at least one processor extracts, using each of the N models, one or more unit waveforms from one set which has not been used to train that model.

6. The information processing apparatus according to claim 4, wherein:
in the extraction process, the at least one processor updates any of the plurality of models using a certain group derived from the plurality of unit waveforms which have been extracted using each of the plurality of models; and
in the extraction process, the at least one processor extracts, using a model which has been updated, one or more unit waveforms from another group derived from the plurality of unit waveforms which have been extracted using each of the plurality of models.

7. The information processing apparatus according to claim 6, wherein:
in the extraction process, the at least one processor carries out updating of a model and extraction using the model two or more times while altering a certain group derived from the plurality of unit waveforms which have been extracted using each of the plurality of models.

8. The information processing apparatus according to claim 1, wherein:
in the extraction process, the at least one processor extracts a unit waveform for which an evaluation value obtained by the model is equal to or greater than a predetermined threshold from among a plurality of unit waveforms included in the another group.

9. The information processing apparatus according to claim 1, wherein:
in the extraction process, the at least one processor extracts a unit waveform for which an evaluation value obtained by the model is greatest from among a plurality of unit waveforms included in the another group.

10. The information processing apparatus according to claim 1, wherein:
in the training data generation process, the at least one processor generates an added unit waveform by adding up a plurality of unit waveforms extracted in the extraction process, and generates the training data including the added unit waveform which has been generated.

11. The information processing apparatus according to claim 1, wherein:
the at least one processor further carries out an evaluation model training process of training an evaluation model using the training data which has been generated in the training data generation process, the evaluation model including the model or being different from the model.

12. The information processing apparatus according to claim 11, wherein:
the at least one processor further carries out an evaluation process of evaluating, using the evaluation model which has been trained in the evaluation model training process, a plurality of unit waveforms that are obtained from evaluation electrocardiogram data for evaluation; and
the at least one processor further carries out an evaluation integration process of evaluating the evaluation electrocardiogram data with reference to evaluation in the evaluation process with respect to the plurality of unit waveforms.

13. The information processing apparatus according to claim 12, wherein:
in the evaluation integration process, the at least one processor evaluates the electrocardiogram data as a disease in a case where one or more unit waveforms which have been evaluated as a disease in the evaluation process include a unit waveform for which an evaluation value obtained in the evaluation process is equal to or greater than a predetermined value, or in a case where the number of unit waveforms which have been evaluated as a disease in the evaluation process is equal to or greater than a predetermined value.

14. The information processing apparatus according to claim 13, in which:
the at least one processor further carries out a first display process of displaying an evaluation result obtained in the evaluation integration process.

15. The information processing apparatus according to claim 1, wherein:
the at least one processor further carries out a second display process of displaying at least one of the plurality of unit waveforms which have been obtained by dividing the waveform indicated by the electrocardiogram data or at least one of the one or more unit waveforms which have been extracted in the extraction process.

16. An information processing method, comprising:
acquiring, by at least one processor, a plurality of unit waveforms which have been obtained by dividing a waveform indicated by electrocardiogram data;
extracting, by the at least one processor, using a model which has been trained with use of a certain group derived from the plurality of unit waveforms, one or more unit waveforms from another group derived from the plurality of unit waveforms; and
generating, by the at least one processor, training data including the one or more unit waveforms which have been extracted.

17. A computer-readable non-transitory storage medium storing a program for causing a computer to function as an information processing apparatus recited in claim 1, the program causing the computer to carry out the acquisition process, the extraction process, and the training data generation process.
